(19) United States
(12) Patent Application Publication
(10) Pub. No.: US 2024/0335490 A1
(43) Pub. Date: Oct. 10, 2024

(54) COMPOSITION FOR PREVENTING OR AMELIORATING LOCOMOTIVE SYNDROME

(71) Applicant: DIC Corporation, Tokyo (JP)

(72) Inventors: Yurino KOSEKI, Chiba (JP); Yasuyuki IMAI, Chiba (JP)

(73) Assignee: DIC Corporation, Tokyo (JP)

(21) Appl. No.: 18/681,862

(22) PCT Filed: Jul. 28, 2022
(86) PCT No.: PCT/JP2022/029050

§ 371 Date: Feb. 7, 2024

(30) Foreign Application Priority Data
Aug. 12, 2021 (JP) 2021-131611

(51) Int. Cl. A61K 36/02; A61K 31/4015; A61K 31/7008; A61K 31/728; A61K 36/324; A61K 38/39; A61P 19/02; A61P 21/00

(52) U.S. Cl. CPC A61K 36/02; A61K 31/4015; A61K 31/7008; A61K 31/728; A61K 36/324; A61K 38/39; A61P 19/02; A61P 21/00

(57) ABSTRACT

An object of the present invention is to provide a composition for preventing or ameliorating locomotive syndrome in which disorders of the locomotive organs result in a decline in mobility to increase risk of a state requiring nursing care. The present invention relates to a composition for preventing or ameliorating locomotive syndrome, a composition for suppressing arthralgia, a composition for suppressing a decline in mobility, or a food composition, all of which contain any one or more of spirulina or phycocyanin.

### TECHNICAL FIELD

The present invention relates to a composition for preventing or ameliorating locomotive syndrome in which disorders of the locomotive organs result in a decline in mobility to increase risk of a state requiring nursing care.

### BACKGROUND ART

In recent years, due to population aging in the world, attention has been paid to various functional health foods including foods for specified health uses, and as an option of health promotion and health maintenance that do not depend on pharmaceuticals, the market expands. With this trend, consumer needs are diversified, and health foods promoting various functionalities are developed. In particular, development of a product capable of alleviating a symptom that reduces the behavioral activation of the elderly, such as locomotive syndrome in which with aging, the functions of the locomotive organs such as muscles, bones, and nerves decline to reduce the locomotion ability, causing problems in daily life and frailty in which a symptom advances resulting in health disorders, is demanded.

A lipase activity inhibitor containing phycocyanin as an active ingredient has been reported (see PTL 1) in that spirulina (spirulina extract), or phycocyanin derived from spirulina, has an action of inhibiting the activities of lipases such as pancreatic lipase. In addition, a serum lipid reducing agent containing phycocyanin as an active ingredient has been reported (see PTL 2) in that phycocyanin derived from spirulina has a higher serum lipid ameliorating action than soybean protein has.

### CITATION LIST

### Patent Literature

PTL 1: Japanese Unexamined Patent Application Publication No. 2004-359638
PTL 2: Japanese Unexamined Patent Application Publication No. 2003-137805

### SUMMARY OF INVENTION

### Technical Problem

However, it is not known that ingestion of a composition containing spirulina or phycocyanin alleviates the progression of locomotive syndrome and produces an effect of maintaining and ameliorating mobility.

An object of the present invention is to provide a composition that can be routinely and continuously ingested, prevent locomotive syndrome, ameliorate and alleviate the progression of a condition, and produce an effect of maintaining and ameliorating mobility.

### Solution to Problem

The present inventors have intensively studied to achieve the object, and as a result, confirmed that spirulina and phycocyanin have an effect on the joint and a mobility ameliorating effect, thereby found an effect of preventing and ameliorating locomotive syndrome. Thus, the present invention has been completed.

That is, the present invention includes the following aspects.
[1] A composition for preventing or ameliorating locomotive syndrome containing any one or more of spirulina or phycocyanin.
[2] The composition for preventing or ameliorating locomotive syndrome according to [1], further containing one or more selected from the group consisting of N-acetylglucosamine, chondroitin, a Boswellia serrata extract, hyaluronic acid, and collagen.
[3] The composition for preventing or ameliorating locomotive syndrome according to [1] or [2], wherein the composition being a composition for suppressing arthralgia.
[4] The composition for preventing or ameliorating locomotive syndrome according to any one of [1] to [3], wherein the composition being a composition for suppressing a decline in mobility.
[5] The composition for preventing or ameliorating locomotive syndrome according to any one of [1] to [4], wherein the composition being a food composition.

### Advantageous Effects of Invention

The present invention can provide a composition that can be routinely and continuously ingested, ameliorates locomotive syndrome, alleviates and prevents the progression of a condition, and has an effect of maintaining and ameliorating the mobility.

### DESCRIPTION OF EMBODIMENTS

Hereinafter, a composition according to an embodiment will be described in detail. However, the following description of constituent elements is an example of an embodiment of the present invention, and the present invention is not limited to the contents.

The composition according to the embodiment relates to a composition for preventing or ameliorating locomotive syndrome, the composition containing any one or more of spirulina or phycocyanin.

Hereinafter, the active ingredient of the composition according to the embodiment will be first described, and the application and form of the composition, and the like will be described.

### <Composition for Preventing or Ameliorating Locomotive Syndrome>

The present invention relates to a composition for preventing or ameliorating locomotive syndrome. Locomotive syndrome herein refers to a condition in which the functions of locomotive organs such as muscles, bones, joints, and nerves decline due to aging and the like. Examples of the cause of a decline in the functions of locomotive organs include the condition of sarcopenia that includes a reduction in muscle mass, osteoporosis causing a reduction in bone mass, bone fracture, gonarthrosis, and spondylosis deformans. When locomotive syndrome advances, unsupported daily life activities are difficult and risk requiring nursing care increases, which may result in a condition that is called frailty in which health disorders are likely to occur.

The composition for preventing or ameliorating locomotive syndrome of the present invention produces an effect of preventing or ameliorating various conditions caused by locomotive syndrome, such as a reduction of joint pain in walking and a mobility ameliorating effect, as described in Examples below.

### <Spirulina>

Spirulina used in the present invention refers to minute spiral algae that belong to the genus Spirulina of the family Oscillatoriaceae of the order Nostocales of blue-green algae, and includes an abundant protein, a saccharide, various vitamins, a mineral, and a vegetable pigment.

Examples of the spirulina include Spirulina platensis, Spirulina maxima, Spirulina geitleri, Spirulina siamese, Spirulina major, Spirulina subsalasa, Spirulina princeps, Spirulina laxissima, Spirulina curta, and Spirulina spirulinoides. Among them, examples of the Spirulina platensis that are particularly preferable because they can be artificially cultured and are easily available, include spirulina geitleri, and Spirulina siamese.

As spirulina used for the composition according to the present embodiment, although naturally obtained spirulina or algae (wet algae) in a state of being cultured in a liquid medium may be used as it is, preferably, a spirulina extract that is an extract liquid extracted from spirulina wet algae with a solvent such as water or ethanol or an extract obtained by concentrating or drying the extract liquid is used.

### <<Spirulina Extract>>

An extract liquid used in production of a spirulina extract is not particularly limited as long as the effects of the present invention can be achieved. For example, hot water can be used. In the present embodiment, an extract liquid extracted from spirulina algae with hot water or an extract obtained by concentrating or drying the extract liquid can be preferably used.

A method for obtaining the spirulina extract is not particularly limited. The spirulina extract can be obtained in accordance with an ordinary method. Examples of the method include methods for producing an extract liquid described in NTL 1 and Japanese Unexamined Patent Publication No. H8-9940. Specific examples thereof include a production method described below.

### <<Method for Producing Spirulina Extract>>

The spirulina extract can be produced as a spirulina extract liquid by extraction from spirulina algae with hot water at a temperature exceeding 100° C., adjusting the pH of the extract liquid to a specific acidic condition, and then removing the insoluble fraction.

When the spirulina extract is used in a liquid state, the spirulina extract liquid obtained as described above can be used as it is. A powder obtained by concentrating or drying the spirulina extract liquid can also be used. A spirulina extract used for the composition according to the present embodiment may be a spirulina extract liquid or one produced by concentrating or drying the spirulina extract liquid.

As spirulina for production of the spirulina extract, commercially available spirulina may be used, or spirulina cultured by oneself may be used. Raw spirulina may be used, or spirulina obtained by drying raw spirulina may be used.

Spirulina can be cultured in accordance with a general method used for cultivation of blue-green algae as a cultivation method. For example, spirulina can be cultured and proliferated outsides under a basic condition.

Spirulina obtained by culturing (hereinafter also referred to as spirulina algae) can be used as it is, or cultured spirulina may be collected by filter cloth or filter paper, washed with water, and then suspended in water to obtain a suspension. Furthermore, a culture medium or the suspension may be concentrated to obtain wet algae, the wet algae may be dried by lyophilization, spray-drying (spray-drying), or the like, to obtain dried algae, or the dried algae may be powdered.

The spirulina algae used in the hot water extraction may be any of wet algae, freeze-dried algae, spray-dried algae, or crushed algae. To obtain the crushed algae, for example, a treatment of crushing algae by a general method, for example, an industrial high-pressure pressing method using a French press, may be used.

Next, an operation of hot water extraction will be described. For example, spirulina algae processed as described above is suspended in an extraction solvent such as distilled water in a pressurized container in advance. The suspension concentration is not particularly limited, but is preferably 1 to 20% by mass relative to the solvent in consideration of extraction efficiency, cost during recovery, and the like. The extraction solvent may be tap water, but is preferably distilled water in consideration of application of the extract liquid to food product ingredients. The extraction temperature is usually a temperature exceeding 100° C., preferably 105° C. to 140° C., and more preferably 110° C. to 130° C. The pressure during the extraction is preferably 1.0 to 2.5 atmospheric pressure. During the extraction, a stirring operation may or may not be performed, but the stirring operation is preferably performed from the viewpoint of thermal efficiency. As the extraction time is longer, the extraction amount is larger. From the viewpoint of efficiency, the extraction time is usually preferably 0.5 to 4 hours.

Subsequently, the algal residue and a protein aggregated by thermal denaturation are removed from the algal residue suspension (PH=about 6.8 to 7.0) after the extraction operation. As a removal operation, for example, an operation such as centrifugation or filtration of the suspension may be performed. By this operation, a supernatant is obtained. Since in the supernatant, a large amount of protein is still dissolved, the dissolved protein is also preferably removed. To further remove the dissolved protein, an acid is added to the suspension to adjust the pH of the extract liquid to an acidic condition in which the pH is equal to or less than the isoelectric point of the protein. As a result, the protein is aggregated. The aggregated protein can be separated by centrifugation, filtration, or the like, to obtain the spirulina extract. Alternatively, the pH of the algal residue suspension after the extraction operation may be first adjusted to a pH equal to or less than the isoelectric point of the protein, as described above, without removing the algal residue and the like, and the algal residue and the aggregated protein may be separated by centrifugation, filtration, or the like, similarly. By removing the aggregated protein, the spirulina extract containing polysaccharides in a high yield can be obtained.

In the present invention, the amount of spirulina added is not particularly limited as long as the effects can be achieved. In the composition for preventing or ameliorating locomotive syndrome, the amount of spirulina added in terms of dry weight is preferably 10% by mass or more, and more preferably 15% by mass or more since the locomotive syndrome ameliorating effect is significantly obtained.

The daily intake of spirulina is not particularly limited as long as the effects of the present invention can be achieved. The daily intake of spirulina is suitably 200 to 100,000 mg, and preferably 500 to 6,000 mg since a joint pain reducing effect is significant.

### <Phycocyanin>

Phycocyanin is one active ingredient in the composition according to the present embodiment. Phycocyanin is a chromoprotein and has phycocyanobilin as a chromophore. Phycocyanin has a structure in which phycocyanobilin is bound to a protein.

Examples of the phycocyanin used in the composition according to the present embodiment include phycocyanin derived from algae, such as phycocyanin derived from blue-green algae, phycocyanin derived from red algae, and phycocyanin derived from cryptophytes. Among them, phycocyanin derived from blue-green algae is preferred because it can be collected in a large amount.

Examples of the blue-green algae include cyanobacteria of the genus Spirulina, Arthrospira, Aphanizomenon, Fisherella, Anabaena, Nostoc, Synechocystis, Synechococcus, Tolypothrix, Aphanothece, Mastigoclaus, and Pleurocapsa. Among them, blue-green algae of the genus Spirulina and Arthrospira, which are produced in an industrial scale and confirmed to be safe, are preferable, and blue-green algae of the genus Spirulina are more preferable.

As a raw material for preparation of phycocyanin, raw blue-green algae may be used, or dried blue-green algae may be used. As a dried product of blue-green algae, a dried product formed from raw blue-green algae in accordance with an ordinary method or a commercially available dried product may be used.

Examples of the phycocyanin include C-phycocyanin, R-phycocyanin, and allophycocyanin. From the viewpoint of quality, safety, availability, or the like, the composition preferably contains C-phycocyanin as phycocyanin. Examples of a preferred aspect of the composition according to the embodiment include the composition containing C-phycocyanin as an active ingredient. Examples of the preferred aspect of the composition according to the embodiment further include the composition containing C-phycocyanin and allophycocyanin. For example, the composition may contain a phycocyanin mixture including C-phycocyanin and allophycocyanin that are obtained by extraction from blue-green algae of the genus Spirulina.

Phycocyanin can be obtained, for example, by suspending blue-green algae in water or a buffer solution such as a phosphate buffer solution or a citric acid buffer and extracting phycocyanin in the blue-green algae.

A method for extracting phycocyanin is not particularly limited. Phycocyanin can be extracted in accordance with an ordinary method.

In a preferred aspect of the extraction method, for example, an extraction method described in Japanese Unexamined Patent Application Publication No. 2006-230272 and the like can be adopted. Specific examples thereof include an extraction method described in the following extraction method (i). By the extraction method (i), high-purity phycocyanin with a vivid hue can be obtained.

<<Method for Extracting Phycocyanin (i)>>

The extraction method (i) includes a first step of extracting phycocyanin in blue-green algae into an aqueous suspension to obtain an extract liquid, a second step of allowing a calcium salt to react with a phosphate in the extract liquid to produce calcium phosphate, and at the same time, allowing the calcium phosphate to adsorb a phycocyanin contaminant to obtain an adsorption product, and a third step of removing the blue-green algae residue and the adsorption product from the extract liquid.

The following extraction method (ii) is more preferable than the extraction method (i).

<<Method for Extracting Phycocyanin (ii)>>

The extraction method (ii) includes a first step of extracting phycocyanin in blue-green algae into an aqueous suspension to obtain an extract liquid, a second step of allowing a calcium salt to react with a phosphate in the extract liquid to produce calcium phosphate, and at the same time, allowing the calcium phosphate to adsorb a phycocyanin contaminant to obtain an adsorption product, a third step of removing the blue-green algae residue and the adsorption product from the extract liquid, and a step of adding a chelating agent to the extract liquid prior to the third step.

By using the method for extracting phycocyanin (i) or (ii), high-quality phycocyanin can be extracted from blue-green algae.

By using the method for extracting phycocyanin (i) or (ii) especially for blue-green algae of the genus Spirulina, high-quality phycocyanin in which C-phycocyanin and allophycocyanin are mixed at a favorable mixing ratio can be extracted.

In the extraction method, the mixing ratio of C-phycocyanin and allophycocyanin may be adjusted within a desired range by selecting an extraction condition as appropriate.

In terms of type, the whole phycocyanin may be C-phycocyanin. Alternatively, the phycocyanin may also contain allophycocyanin, and thus the composition may contain a mixture of C-phycocyanin and allophycocyanin.

For example, the mixing ratio by mass of C-phycocyanin to allophycocyanin is preferably 3 to 9.5:0.5 to 7, more preferably 6 to 9.5:0.5 to 4, and still more preferably 7 to 8:2 to 3.

Although in the present invention, the amount of phycocyanin added is not particularly limited as long as the effects can be achieved, in the composition for preventing or ameliorating locomotive syndrome, the amount of phycocyanin added in terms of dry weight is preferably 0.1% by mass or more, and more preferably 1.0% by mass or more because the locomotive syndrome ameliorating effect is significantly obtained.

Although the daily intake of phycocyanin is not particularly limited as long as the effects of the present invention can be achieved, the daily intake of phycocyanin is suitably 5 to 1,000 mg, and preferably 15 to 5,000 mg because the joint pain reducing effect is significant.

In addition to spirulina described above, the composition for ameliorating locomotive syndrome of the present invention more preferably contains one or more types of animal- or vegetable-derived raw materials, such as N-acetylglucosamine, chondroitin, hyaluronic acid, and a Boswellia serrata extract as an addition ingredient. As the ingredients, commercially available materials and the like can be used, and the ingredients may be added as appropriate to enhance the effects of the present invention. Among them, use of N-acetylglucosamine and chondroitin is preferable since the locomotive syndrome ameliorating effect is enhanced.

### <N-Acetylglucosamine>

N-acetylglucosamine used as the addition ingredient in the present invention is a monosaccharide in which a hydroxyl group at position 2 of glucose is substituted by an acetylamino group. N-acetylglucosamine used in the present invention is not particularly limited as long as the effects of the present invention can be achieved, and a commercially available N-acetylglucosamine or N-acetylglucosamine appropriately modified by a chemical treatment or the like may be used.

Although when in the present invention, N-acetylglucosamine is added, the addition amount is not particularly limited as long as the effects of the present invention can be achieved, the addition amount is preferably 20 to 90% by mass, and preferably 30 to 80% by mass in the composition because a more significant effect on the joint is obtained.

Although the daily intake of N-acetylglucosamine is not particularly limited, the daily intake of N-acetylglucosamine is suitably 300 to 1,500 mg, and preferably 400 to 600 mg because the joint pain reducing effect is significant.

### <Chondroitin>

Chondroitin used as the addition ingredient in the present invention is one type of mucopolysaccharide that is present in the human body in the form of chondroitin sulfate. Chondroitin used in the present invention is not particularly limited as long as the effects of the present invention can be achieved, and a sulfate salt, a sodium salt, or the like may be used. Commercially available chondroitin may be used, and is not particularly limited.

Although when chondroitin is added in the present invention, the addition amount is not particularly limited as long as the effects of the present invention can be achieved, the addition amount is preferably 0.01 to 10% by mass in the composition, and preferably 1 to 7% by mass because a significant effect on the joint is obtained.

Although the daily intake of chondroitin is not particularly limited, the daily intake of chondroitin is suitably 5 to 1,500 mg, and preferably 15 to 30 mg because the joint pain reducing effect is significant.

### <Hyaluronic Acid>

Hyaluronic acid used as the addition ingredient in the present invention is one type of linear mucopolysaccharide. Hyaluronic acid used in the present invention is not particularly limited as long as the effects of the present invention can be achieved, and a sodium salt or the like may be used. Commercially available hyaluronic acid may be used, and there is no particular limitation.

Although when in the present invention, hyaluronic acid is added, the addition amount is not particularly limited as long as the effects of the present invention can be achieved, the addition amount is preferably 0.01 to 10% by mass, and preferably 1 to 5% by mass in the composition because a more significant effect on the joint is obtained.

Although the daily intake of hyaluronic acid is not particularly limited as long as the effects of the present invention can be achieved, the daily intake of chondroitin is suitably 10 to 240 mg, and preferably 20 to 120 mg because the joint pain reducing effect is significant.

<Boswellia serrata Extract>

A Boswellia serrata extract used as the additive ingredient in the present invention is an extract, the raw material for which is Indian frankincense. The Boswellia serrata extract used in the present invention is not particularly limited as long as the effects of the present invention can be achieved, and a Boswellia serrate extract produced by various extraction methods or processing methods may be used.

Although when in the present invention, the Boswellia serrata extract is added, the addition amount is not particularly limited as long as the effects of the present invention can be achieved, the addition amount is preferably 1.0 to 30% by mass, and preferably 5.0 to 20% by mass in the composition because a significant effect on the joint is obtained.

Although the daily intake of the Boswellia serrata extract is not particularly limited, the daily intake of the Boswellia serrata extract is suitably 50 to 200 mg, and preferably 70 to 150 mg because the joint pain reducing effect is significant.

### <Collagen>

Collagen used as the additive ingredient in the present invention is a protein constituting a cartilage, a tendon, a ligament, a dermis, and the like of vertebrates. The collagen used as the additive ingredient in the present invention may be any of animal-derived collagen or synthetic collagen. Furthermore, a collagen protein, a collagen peptide obtained by hydrolysis of a collagen protein, or an atelocollagen obtained by processing a collagen molecule with protease and removing a telopeptide moiety may be used. The animal-derived collagen is preferably fish-derived collagen.

The average molecular weight (weight average molecular weight) of the collagen used in the present invention is not particularly limited, but for example, is preferably 500 to 1,000,000, and more preferably 1,000 to 300,000.

Although when in the present invention, collagen is added, the addition amount is not particularly limited as long as the effects of the present invention can be achieved, the addition amount is preferably 0.1 to 10% by mass, and preferably 0.2 to 5% by mass in the composition because a significant effect on the joint is obtained.

The daily intake of collagen is not particularly limited, but the daily intake of collagen is suitably 0.5 to 100,000 mg, and preferably 1 to 50 mg because the joint pain reducing effect is significant.

The composition for preventing or ameliorating locomotive syndrome of the present invention can be used as a food composition for oral use and a pharmaceutical composition described below.

### (Food Composition)

When a general food in the field of foods and an effective amount of the active ingredient capable of effectively exerting an intended action as food ingredients are blended in various foods, the composition according to the embodiment can be provided as a food composition having the action. The composition according to the embodiment can be suitably used, for example, for compositions of foods such as a health food, a functional food, a dietary supplement, a supplement, a food with health claims, a food for specified health uses, a food with nutrient function claims, a food with functional claims, a food for the sick, a food additive, and a feed and a feed additive. The form of the food composition is not particularly limited, and can be appropriately selected according to purposes, and the composition can be, for example, solid, liquid, gel, or the like.

It is noted that the food with functional claims is a food that is labeled with functionality based on scientific evidence under the business operator's own responsibility, and in which information about the evidence of safety and functionality is submitted to the Secretary-General of the Consumer Affairs Agency before sale. The food composition according to the embodiment may be labeled with “ameliorating trouble with the knee joint during movement”, “for those who are concerned about walking or going up and down the stairs”, “alleviating trouble with the movement of the knee”, “reducing discomfort of the knee”, or other indications as a food composition for preventing or ameliorating locomotive syndrome.

Examples of the food composition include non-alcoholic beverages such as soft drink, carbonated drink, fruit juice, vegetable juice, fruit and vegetable juice, domestic animal milk such as cow milk, soy milk, milk drink, yogurt drink, jelly drink or jelly stick, coffee, cocoa, tea drink, nutrition supplement drink, energy drink, sport drink, mineral water, flavored water, and non-alcoholic beer-flavored drink; carbohydrate-containing foods or beverages such as cooked rice, noodle, bread, and pasta; dairy products such as cheese, hard or soft yogurt, fresh cream derived from domestic animal milk or other oil and fat material, and ice cream; various confectionery including western confectionery such as cookie, cake, and chocolate, Japanese confectionery such as manju and yokan, tablet confectionery (refreshing confectionery) such as ramune candy, candy, gum, frozen dessert such as jelly and pudding, and snack confectionery; alcoholic beverages such as whiskey, bourbon, spirit, liqueur, wine, fruit liqueur, Japanese sake, Chinese liquor, shochu, beer, non-alcoholic beer having an alcohol content of 1% or less, low-malt beer, other liquor, and soda and shochu; processed food products including egg processed foods, processed foods formed of fish and shellfish or meat (including organ meat such as liver) (including a dainty), soups such as miso soup, seasonings such as miso, soy, a flaked seasoning, and another seasoning, and liquid diets such as a concentrated liquid diet.

When the food composition is, for example, a health food, a functional food, a dietary supplement, a supplement, a food with health claims, a food for specified health uses, a food with nutrient function claims, a food with functional claims, a food for the sick, a food additive, a feed and a feed additive, or the like, the food composition may have a tablet (including a chewable tablet or the like), capsule, troche, syrup, jelly, granule, or powder form, or the other form.

In addition to the active ingredient described above, one or two or more types of ingredients capable of being usually used for a food composition may be optionally selected and blended in the food composition according to the embodiment. For example, all additives capable of being usually used in the food field, such as various seasonings, a preservative, an emulsifier, a stabilizer, an excipient, a lubricant, a perfume, a colorant, an antiseptic, and a pH modifier can be blended.

### (Pharmaceutical Composition)

When an effective amount of the active ingredient capable of effectively exerting an intended action in the field of pharmaceuticals, a pharmaceutically acceptable carrier or additive, and the like are blended, the composition according to the embodiment can be provided as a pharmaceutical composition having the action. The composition according to the embodiment can be suitably used, for example, as the pharmaceutical composition for treating, preventing, or ameliorating locomotive syndrome. The pharmaceutical composition may be a pharmaceutical or a quasi drug.

The form of the pharmaceutical composition is not particularly limited, and can be suitably selected according to purposes, and the pharmaceutical composition may be, for example, solid, liquid, gel, or the like.

The pharmaceutical composition may contain an additive such as a carrier, a binder, a stabilizer, an excipient, a diluent, a pH buffer, a disintegrant, a solubilizer, a solubilizing agent, or an isotonic agent that is pharmaceutically acceptable and generally used. The pharmaceutical composition may be for oral use or parenteral use, and is preferably for oral use. The form of the pharmaceutical composition for oral use may be a generally used administration form, for example, a dosage form of a tablet, a powder, a granule, a capsule, a syrup, a suspension, or the like. The form of the pharmaceutical composition for parenteral use may be a generally used administration form, for example, injection (subcutaneous injection, intravenous injection, intramuscular injection, or the like) in the dosage form of a solution, an emulsion, or a suspension, or nasal administration in the form of a spray preparation.

An example in which the form of the food composition and/or the pharmaceutical composition is a tablet will be described below.

When the form of the food composition and/or the pharmaceutical composition is, for example, a pill form (also referred to as a tablet), the active ingredient can be appropriately combined with an additive such as an excipient, a binder, a disintegrant, a lubricant, a preservative, an antioxidant, an isotonic agent, a buffer, a coating agent, a corrigent, a stabilizing agent, a base, a dispersant, a stabilizer, or a colorant, to prepare the food composition and/or the pharmaceutical composition in accordance with an ordinary method.

A method for producing the tableted food or beverage of the present invention is not particularly limited as long as the effects of the present invention can be obtained. The tableted food or beverage is produced by a process including a mixing step of mixing various raw materials and a tableting step.

In the mixing step, mixing using a V-shaped mixer, a W-shaped mixer, or a Bohle container mixer can be employed. In particular, mixing using a V-shaped mixer is preferred.

In the tableting step, tableting using a rotary-type tablet press machine, a single-punch tablet press machine, or a double-punch tablet press machine can be employed. In particular, tableting using a rotary tablet press machine is preferred.

Examples of the excipient include starch and derivatives thereof (dextrin, carboxymethyl starch, etc.), cellulose and derivatives thereof (methyl cellulose, hydroxypropylmethyl cellulose, etc.), saccharides (lactose, saccharose, glucose, trehalose, etc.), citric acid and salts thereof, malic acid and salts thereof, and ethylene diamine tetraacetate and salts thereof.

Examples of the binder include starch and derivatives thereof (pregelatinized starch, dextrin, etc.), cellulose and derivatives thereof (ethylcellulose, carboxymethyl cellulose sodium, hydroxypropylmethyl cellulose, etc.), gum arabic, tragacanth, gelatin, saccharides (glucose, saccharose, etc.), and ethanol.

Examples of the disintegrant include starch and derivatives thereof (carboxymethyl starch, hydroxypropyl starch, etc.), cellulose and a derivative thereof (carboxymethyl cellulose sodium, crystalline cellulose, hydroxypropylmethyl cellulose, etc.), carbonic acid salts (calcium carbonate, calcium hydrogen carbonate, etc.), tragacanth, gelatin, and agar.

Examples of the lubricant include stearic acid, calcium stearate, magnesium stearate, talc, titanium oxide, calcium hydrogen phosphate, dried aluminum hydroxide gel, sucrose fatty acid esters, and edible oils and fats.

Examples of the preservative include a p-hydroxybenzoate ester, sulfurous acid salts (sodium sulfite, sodium pyrosulfite, etc.), phosphoric acid salts (sodium phosphate, calcium polyphosphate, sodium polyphosphate, sodium metaphosphate, etc.), dehydroacetic acid, sodium dehydroacetate, sorbic acid, glycerol, and saccharides.

Examples of the antioxidant include a sulfurous acid salt (sodium sulfite, sodium hydrogen sulfite, etc.), erythorbic acid, L-ascorbic acid, cysteine, thioglycerol, butylhydroxyanisol, dibutylhydroxytoluene, propyl gallate, ascorbic acid palmitate, and dl-α-tocopherol.

Examples of the tonicity agent include sodium chloride, sodium nitrate, potassium nitrate, dextrin, glycerol, and glucose.

Examples of the buffer include sodium carbonate, hydrochloric acid, boric acid, and phosphoric acid salts (sodium hydrogenphosphate).

Examples of the coating agent include cellulose derivatives (hydroxypropyl cellulose, cellulose acetate phthalate, hydroxypropylmethyl cellulose phthalate, etc.), shellac, polyvinyl pyrrolidone, polyvinyl pyridine (poly-2-vinyl pyridine, poly-2-vinyl-5-ethyl pyridine, etc.), polyvinyl acetyl diethyl aminoacetate, polyvinyl alcohol phthalate, and a methacrylate-methacrylic copolymer.

Examples of the taste masking agent include saccharides (glucose, saccharose, lactose, etc.), saccharin sodium, and sugar alcohols.

Examples of the solution adjuvant include ethylenediamine, nicotinic acid amide, saccharin sodium, citric acid, citric acid salts, sodium benzoate, polyvinyl pyrrolidone, polysorbate, sorbitan fatty acid esters, glycerol, propylene glycol, and benzyl alcohol.

Examples of the base include fats (lard, etc.), vegetable oil (olive oil, sesame oil, etc.), animal oil, lanolin acid, vaseline, paraffin, resin, bentonite, glycerol, and glycol oil.

Examples of the dispersant include gum arabic, tragacanth, a cellulose derivative (methyl cellulose etc.), sodium alginate, polysorbate, and sorbitan fatty acid esters.

Examples of the stabilizer include sulfurous acid salts (sodium hydrogen sulfite, etc.), nitrogen, and carbon dioxide.

The total content of the active ingredients in the food composition and/or the pharmaceutical composition according to the embodiment (the content obtained by summating all the contents of the active ingredients contained in the composition) varies depending on conditions such as the type, ingredient, and form of a food or a pharmaceutical, is not particularly limited as long as the effects of the present invention can be achieved, and can be appropriately selected.

In particular, when the form of the food composition and/or the pharmaceutical composition according to the embodiment is a tablet, the whole content of the active ingredient in the tablet is not particularly limited as long as the effects of the present invention can be achieved. In the whole mass of the food composition and/or the pharmaceutical composition, the content of the active ingredient in terms of dry weight is preferably 20% by mass or more, and more preferably 50% by mass or more. The whole content may be 100% by mass or less, and preferably 99% by mass or less.

In the present invention, the total intake of each of the ingredients is not particularly limited as long as the effects of the present invention can be achieved. The amount of each ingredient blended preferably falls within the range of the daily intake.

### Examples

Hereinafter, the present invention will be described in detail with reference to Examples, but the scope of the present invention is not limited to these Examples.

Ingredients were mixed at proportions shown in Table 1 below and subjected to tablet molding to produce tableted products in Examples 1 to 3 and Comparative Examples 1 and 2 in the present invention. As the blended ingredients, commercially available products were used. In order to uniform appearance, Blue No. 2 was added to adjust the color. In each of Examples and Comparative Examples, six tableted products were produced such that the daily intake was 1,800 mg.

### <Method for Confirming Effect>

Twenty male and female subjects who feel a decline in mobility in their 40s to 70s were divided into five groups of four subjects each. In each group, two tablets in each of Examples 1, 2, and 3 and Comparative Examples 1 and 2 were taken three times a day in the morning, noon, and evening for four weeks. Two weeks and four weeks after the initiation of the test, the following questionnaires were carried out.

### <Joint Pain Reducing Effect>

An equal-interval scale of 0 to 10 was prepared, and a score of 5 that is the middle means before the initiation of the test or no change, a score closer to 0 means deterioration, and a score closer to 10 means amelioration. The subjects were made to plot the experience evaluation of the joint pain reducing effect in which the subjects felt in walking. Scores plotted by the subjects were considered to be test evaluation. The averages in each group of the participants two weeks and four weeks after the initiation of the test are shown in Table 1.

### <Mobility Ameliorating Effect>

An equal-interval scale of 0 to 10 was prepared, and a score of 5 that is the middle means no change, a score closer to 0 means that walking is difficult, and a score closer to 10 means that walking is easy. The subjects were made to plot the experience evaluation. Scores plotted by the subjects were considered to be test evaluation. The averages in each group of the participants two weeks and four weeks after the initiation of the test are shown in Table 1.

TABLE 1

| | | | | Comp. | Comp. |
|---|---|---|---|---|---|
| | Example 1 | Example 2 | Example 3 | Example 1 | Example 2 |
| | Blending | Blending | Blending | Blending | Blending |
| | amount | amount | amount | amount | amount |
| | [mg] | [mg] | [mg] | [mg] | [mg] |
| Spirulina | 600 | | | | |
| Phycocyanin | | 60 | 30 | | |
| N-acetylglucosamine | | | 500 | 500 | |
| Boswellia serrata | | | 100 | 100 | |
| extract | | | | | |
| Chondroitin | | | 20 | 20 | |
| Hyaluronic acid | | | 30 | 30 | |
| II type collagen | | | 5 | 5 | |
| Silicon oxide fine | 30 | 30 | 30 | 30 | 30 |
| particles | | | | | |
| Calcium stearate | 30 | 30 | 30 | 30 | 30 |
| Cellulose | 1140 | 1680 | 1055 | 1085 | 1740 |
| Blue No. 2 (toning | As | As | As | As | As |
| agent) | appropriate | appropriate | appropriate | appropriate | appropriate |

Evaluation results

| | | | | | |
|---|---|---|---|---|---|
| Effect on joint (2 | 4.975 | 5 | 5.25 | 5.175 | 5 |
| weeks) | | | | | |
| Effect on joint (4 | 5.7 | 5.95 | 6.25 | 5.85 | 4.825 |
| weeks) | | | | | |
| Mobility ameliorating | 5.525 | 5.85 | 5.6 | 5.2 | 5.025 |
| effect (2 weeks) | | | | | |
| Mobility ameliorating | 6.15 | 6.3 | 6 | 5.3 | 5.025 |
| effect (4 weeks) | | | | | |

From the results of Table 1, spirulina and phycocyanin were recognized to exert the effect on the joint and the mobility ameliorating effect. On the other hand, the composition in Comparative Example 1 was recognized to exert the effect on the joint, but was not recognized to exert the mobility ameliorating effect.

1. A method for preventing or ameliorating locomotive syndrome, comprising:
administrating a composition comprising any one or more of spirulina or phycocyanin.

2. The method according to claim 1, wherein the composition further comprises one or more selected from the group consisting of N-acetylglucosamine, chondroitin, a Boswellia serrata extract, hyaluronic acid, and collagen.

3. The method according to claim 1, wherein the method suppresses arthralgia.

4. The method according to claim 1, wherein the method suppresses a decline in mobility.

5. The method according to claim 1, wherein the composition is a food composition.
